(19) United States
(12) Patent Application Publication
(10) Pub. No.: US 2018/0291910 A1
(43) Pub. Date: Oct. 11, 2018

(54) Methods and Apparatus for an Automated Fluid Pumping System

(71) Applicant: Tigmill Technologies, LLC, Farmers Branch, TX (US)

(72) Inventors: Jerry Mack Mills, Coppell, TX (US); Travis R. Wood, Longview, TX (US); Richard Dale Lee, Longview, TX (US); Howard Lee Tigelaar, Allen, TX (US)

(21) Appl. No.: 15/950,904

(22) Filed: Apr. 11, 2018

Related U.S. Application Data
(60) Provisional application No. 62/484,220, filed on Apr. 11, 2017.

(51) Int. Cl. F04D 15/02; E21B 43/12; E21B 47/00; F04B 47/06; F04C 2/107; F04D 13/10; G01N 33/24

(52) U.S. Cl. CPC F04D 15/0209; E21B 43/121; E21B 47/0007; F04B 47/06; F05B 2270/502; F04D 13/10; G01N 33/246; F05B 2210/13; F05B 2210/20; F04C 2/1071

(57) ABSTRACT

In a described example, an automated fluid pumping system (AFPS) includes a fluid pump coupled to a pump controller, an electronic sensor that detects air, oil, or water coupled to a sensor controller, and the sensor controller coupled to the pump controller. The pump controller is configured to control the operation of the fluid pump based on a detected fluid in the well as determined by the electronic sensor.

### CROSS-REFERENCE TO RELATED APPLICATIONS

This application claims priority to U.S. Provisional Application No. 62/484,220, filed Apr. 11, 2017, which is incorporated herein by reference in its entirety.

### TECHNICAL FIELD

This disclosure relates generally to oil wells, and more particularly to an automated oil recovery system.

### BACKGROUND

Most oil is pumped from oil wells using a pumpjack (also known as a sucker rod pump), a progressive cavity pump (PCP), or an electrical submersible pump (ESP). In a pumpjack a stationary valve closes the lower end of the working tubing deep in the well under the oil. A moving valve that slides inside the working tubing under the oil is attached to the lower end of a sucker rod that runs up the working tubing and is attached to the pump jack at the well head. Reciprocal motion of the pump jack alternately lowers and raises the moving valve. During the downward motion of the moving valve the stationary valve closes and the moving valve opens allowing oil to pass through the moving valve and fill the working tubing above the moving valve. During the upward motion of the moving valve the moving valve closes pushing the oil column above it up and out the top of the well. At the same time the stationary valve opens allowing more oil to be pulled up into the lower end of the working tubing.

An ESP or a PCP is a pump with an electrical motor attached to the bottom of a long flexible production tube or metal pipe also known as working tubing. Electrical power is provided from a pump controller at the well head to the ESP or PCP with a power cord either banded to the working tubing or incorporated in the working tubing when the working tubing is manufactured.

One problem that is common to pumps is the damage that may occur if the pump runs out of fluid and begins pumping dry. This results in costly well workover and pump repair or replacement.

Also common when pumping oil is that the water table may rise so that water instead of oil is pumped. Water pumped from deep in the earth is highly contaminated with various minerals and salts and is expensive to dispose. It is desirable to reduce the amount of water pumped during oil recovery.

### SUMMARY

In a described example, an automated fluid pumping system (AFPS) includes a fluid pump coupled to a pump controller, and an electronic sensor that detects air, oil, and/or water coupled to a sensor controller with a relay and a microprocessor, where the sensor controller coupled to the pump controller. In a described example, a method for operating an AFPS includes pumping fluid until the electronic sensor detects the absence of fluid, turning the pump off, after a time delay turning the pump back on, pumping until the electronic sensor again detects the absence of fluid and repeating the steps. In a described example, a method for operating an AFPS includes pumping fluid until a lower electronic sensor detects the absence of fluid, turning the pump off allowing the fluid column to recover, an upper electronic sensor detecting the fluid column and resuming pumping until the first electronic sensor again detects the absence of fluid and repeating the steps. In a described example, a method for operating an AFPS includes pumping fluid until an electronic sensor detects the absence of fluid, turning the pump off allowing the fluid column to recover, a pressure sensor detecting the recovered fluid column and resuming pumping until the first electronic sensor again detects the absence of fluid and repeating the steps.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 is an illustration of an automated fluid pumping system in accordance with some embodiments;

FIG. 2 is a block diagram of a sensor controller in accordance with some embodiments;

FIG. 3 is a flow diagram describing the operation of an automated fluid pumping system in accordance with some embodiments;

FIG. 4 is an illustration of an automated fluid pumping system in accordance with some embodiments;

FIG. 5 is a block diagram of a sensor controller in accordance with some embodiments;

FIG. 6 is a flow diagram describing the operation of an automated fluid pumping system in accordance with some embodiments;

FIG. 7 is an illustration of an automated fluid pumping system in accordance with some embodiments;

FIG. 8 is a block diagram of a sensor controller in accordance with some embodiments;

FIG. 9 is a flow diagram describing the operation of an automated fluid pumping system in accordance with some embodiments; and

FIG. 10 is a block diagram of a controller in accordance with some embodiments.

### DETAILED DESCRIPTION OF ILLUSTRATIVE EMBODIMENTS

Corresponding numerals and symbols in the different figures generally refer to corresponding parts unless otherwise indicated. The figures are not necessarily drawn to scale.

The making and using of embodiments of this disclosure are discussed in detail below. It should be appreciated, however, that the concepts disclosed herein can be embodied in a wide variety of specific contexts, and that the specific embodiments discussed herein are merely illustrative and do not serve to limit the scope of the claims. Further, it should be understood that various changes, substitutions and alterations can be made herein without departing from the spirit and scope of this disclosure as defined by the appended claims.

An automated fluid pumping system (AFPS) integrates an electronic fluid sensor and sensor controller with a pump controller. An example is an automated well fluid pumping system (AWPS). In some embodiments, the electronic fluid sensor is configured to detect the presence or absence of fluid in a well, and/or may be configured to determine the composition of a detected fluid in the well. The electronic fluid sensor may detect the presence or absence of fluid, or the composition of a detected fluid, electronically with no moving parts. In some embodiments, the fluid sensor can detect oil or can detect water, and can determine if the fluid sensor is in water, in oil, in air, and/or a combination thereof. Additional sensors that provide additional capability such as temperature, pressure, amperage, and conductivity can be integrated with the electronic fluid sensor in some embodiments. For example, multiple sensors may be physically packaged together, and/or may share some physical components such as a power supply or electrical cables. The electronic fluid sensor may be, for example, a sensor that is described in U.S. patent application Ser. No. 14/817,409 filed Aug. 4, 2015, and/or U.S. patent application Ser. No. 15/821,520, filed Nov. 22, 2017. Both applications are incorporated herein in their entirety for reference. The electronic fluid sensor may interact with the pump controller in the AFPS to control the AFPS equipment. In some embodiments, the AFPS may reduce the time a pumper needs to spend at the well, and also protect the pump from damage due to pump off.

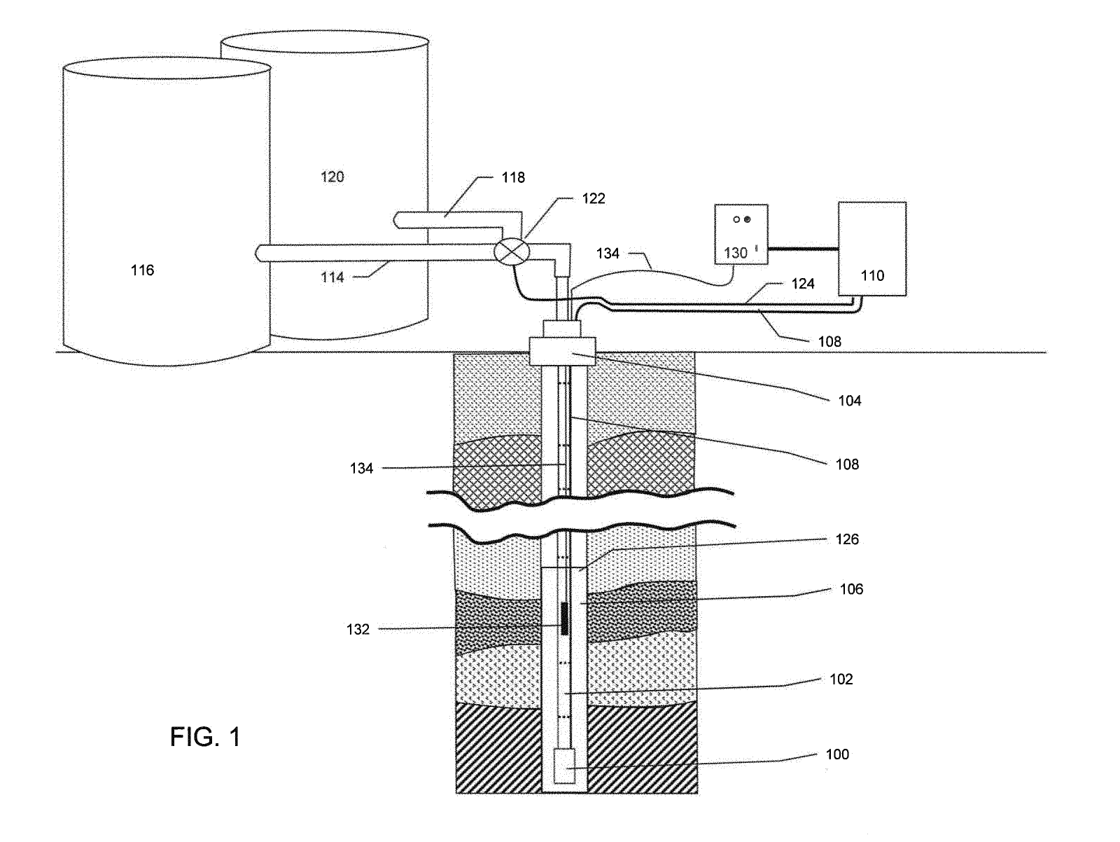

FIGS. 1, 2, and 3 respectively illustrate an embodiment AFPS, a sensor controller which may be used in an embodiment AFPS, and an embodiment of a method for operating an embodiment AFPS. In FIGS. 1, 2, and 3 an electrical submersible pump (ESP) is used for illustration, but a progressive cavity pump or a pumpjack or other suitable pump could also be used.

FIG. 1 depicts an AFPS in accordance with some embodiments. A first pipe 114 feeds an oil storage tank 116 and a second pipe 118 feeds a water storage tank 120. The oil storage tank 116 may be used to store oil, and the water storage tank 118 may be used to store water. The oil storage tank 116 and the water storage tank 120 may be placed proximate to a surface of an oil well, where a well head 104 is located at the surface of the oil well. A valve 122 is connected to first pipe 114, second pipe 118, and well head 104. In some embodiments valve 122 can direct the fluid flow from the well head 104 to either oil storage tank 116 and/or water storage tank 120. Alternatively, an oil/water separator (not shown in FIG. 1), also known as a gun barrel separator, may be inserted between the well head 104 and the oil storage tank 116 and water storage tank 120, to separate oil from an oil/water mixture.

A ESP 100, or another suitable pump, is placed in the well. The ESP 100 is connected to well head 104 by a working tube 102. In operation, the ESP 100 pumps fluid 106 from the well, through the working tube 102, to the well head 104.

A pump controller 110 is disposed at the surface of the oil well. The pump controller 110 contains electronics to control components of the AFPS. For example, the pump controller 110 may be configured to receive information from sensors located on equipment of the AFPS, to send power to the ESP 100, to turn the ESP 100 off and on, to control the valve 122, to turn other components on and off, and the like. The pump controller 110 may also contain wireless or Bluetooth communications equipment to enable a remote user to communicate with and/or control equipment of the well from a remote location. Cable 108 extends from the pump controller 110 into the oil well and connects to ESP 100. In some embodiments cable 108 conveys power to the ESP 100 from pump controller 110, and in some embodiments may also provide communication between sensors in the fluid and the pump controller 110. Cable 124 extends from the pump controller 110 to the valve 122, and in some embodiments enables the pump controller 110 to control the position of the valve 122.

The AFPS may also include a sensor system, which in some embodiments includes an electronic sensor 132 and a sensor controller 130. The sensor system may be utilized in the AFPS to indicate to the pump controller 110 the presence or absence of, or composition of, fluids such as oil and/or water in the well. In some embodiments, sensor system may provide additional well data to the pump controller 110, such as well temperature and fluid pressure from other integrated sensors if present.

In some embodiments, the electronic sensor 132 is placed in the well, for example attached to the outside of the working tube 102 between the working tube 102 and the well casing. The sensor controller 130 may be disposed at the surface of the oil well, and may be physically and/or electrically coupled to the pump controller 110. In some embodiments, a single controller may implement the sensor controller 130 and the pump controller 100 described herein. The electronic sensor 132 is connected to the sensor controller 130 by a cable 134. During operation, the electronic sensor 132 and the sensor controller 130, alone or in combination, may be configured to detect a composition of a fluid 106 in which the electronic sensor 132 is submerged, and/or may be configured to detect the presence or absence of a fluid 106 at the depth at which the electronic sensor 132 is disposed. Data from the electronic sensor 132 may be transmitted over cable 134 to sensor controller 130. The data may be used to detect a composition of a fluid 106 in which the electronic sensor 132 is submerged, and/or may be configured to detect the presence or absence of a fluid 106 at the depth at which the electronic sensor 132 is disposed. The determined fluid composition, or the determined presence or absence of fluid 106, may be transmitted to pump controller 110. Pump controller 110 may use the data from electronic sensor 132 and/or sensor controller 130 to control equipment of the AFPS.

For example, in some embodiments the electronic sensor 132 and/or the sensor controller 130 may detect a change in composition of the fluid 106 in which the electronic sensor 132 is disposed. The electronic sensor 132 and/or the sensor controller 130 may detect a change in the fluid 106 in which the electronic sensor 132 is disposed from oil to water, or from water to oil. In response to detecting the change in the fluid 106, the sensor controller 130 can send a signal to the pump controller 110, which, in response to receiving the signal from sensor controller 130, may send a signal to the valve 122 to instruct the valve 122 to position itself to direct the fluid 106 from the oil well (for example in working tube 102) to the appropriate oil storage tank 116 or water storage tank 120, depending on the composition of the detected fluid 106. Examples of cable 134 that can be used include a mono-conductor shielded cable or a shielded twisted pair to provide a signal lead and a ground to the electronic sensor 132.

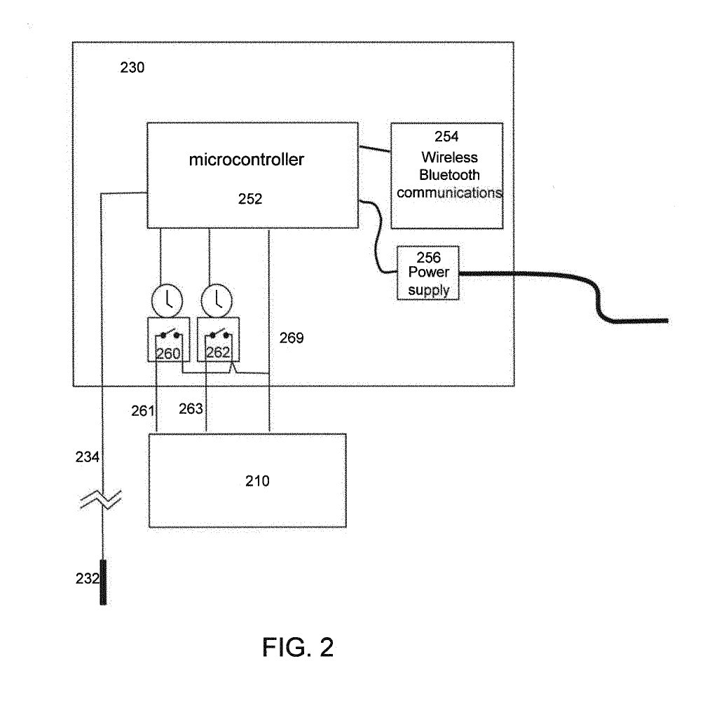

FIG. 2 depicts a sensor controller 230 in accordance with some embodiments. In FIG. 2, elements designated as 2xx correspond to elements designated as 1xx in FIG. 1. For example, sensor controller 230 in FIG. 2 corresponds to the sensor controller 130 in FIG. 1.

Sensor controller 230, as shown in FIG. 2, may be used to implement sensor controller 130 as described in connection with FIG. 1. Sensor controller 230 includes a microcontroller 252, power supply 256, an optional wireless Bluetooth communications module 254, and a plurality of relays, such as oil sensor relay 260 and water sensor relay 262. Cable 234 connects the sensor controller 230 to an electronic sensor 232, which may be disposed between a working tube 102 and a well casing of an oil well (for example depicted as electronic sensor 132 as shown in FIG. 1). Power supply 256 may provide power to microcontroller 252. In some embodiments, power supply 256 provides low voltage power to the microcontroller 252. In some embodiments, the low voltage power that is provided may be less than about 30 V.

The microcontroller 252 may receive signals from the electronic sensor 232 through cable 234. A plurality of relays may be present in sensor controller 230, such as an oil sensor relay 260 and a water sensor relay 262. Although two relays are shown in FIG. 2, in other embodiments fewer relays or additional relays may be present. Oil sensor relay 260 and water sensor relay 262 are electrically connected to the microcontroller 252. The oil sensor relay 260 and water sensor relay 262 are also electrically connected to pump controller 210. In some embodiments, oil sensor relay 260 is connected to pump controller 210 by conductor 261 and water sensor relay 262 is connected to pump controller 210 by conductor 263. A conductor also connects microcontroller 252 directly to pump controller 210. For example, ground conductor 269 connects the sensor controller 230 to the pump controller 210.

As is illustrated in FIG. 2, in some embodiments the oil sensor relay 260 and the water sensor relay 262 can be time delay relays that reset themselves after a predetermined interval. In other embodiments, the oil sensor relay 260 and the water sensor relay 262 can be reset by the microcontroller 252. The time delay can be fixed, can be physically reprogrammed by a pumper, can be electronically reprogrammed by the microcontroller 252, or the like.

In some embodiments, information regarding the status of the oil relay 260 and the water relay 262 can be transmitted wirelessly to the pump controller 210 using an optional wireless or Bluetooth communications module 254. In such a case, conductors 261, 263, and conductor 269 may be unnecessary.

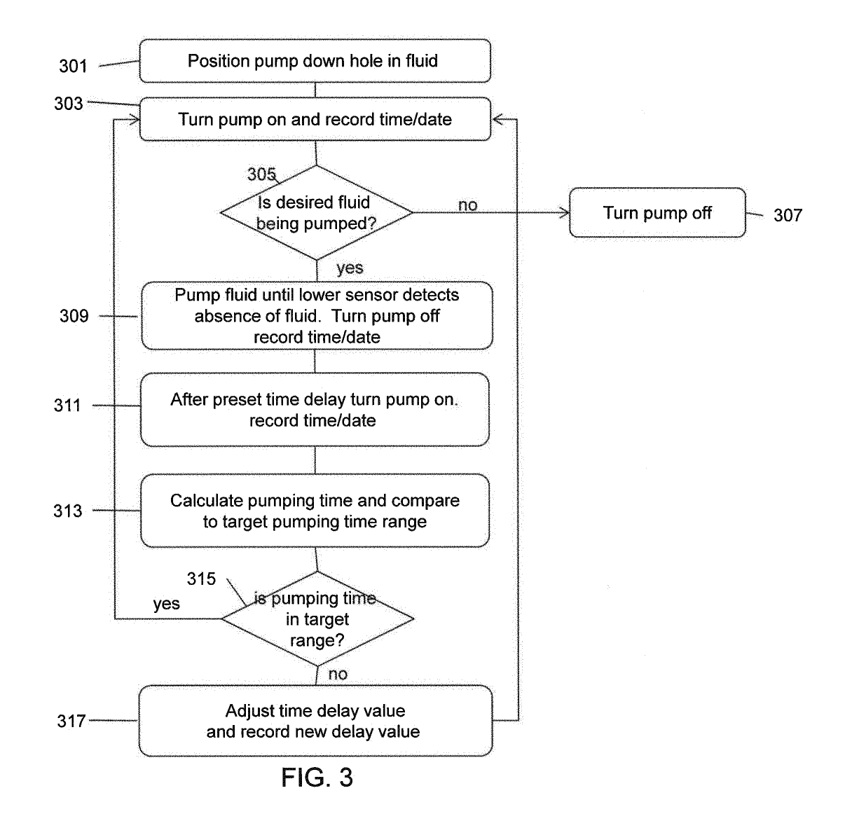

Turning to FIG. 3, a process of operating an automated oil recovery system in accordance with some embodiments is illustrated. In describing the process of FIG. 3, the referenced components are shown in FIG. 1 unless otherwise indicated.

In step 301 the ESP 100 (or other suitable pump) is submersed in a fluid 106, for example, down hole in the well.

In step 303 the pump 100 is turned on and the time and date are recorded. For example, the pump controller 110 may automatically turn on the pump 100 and the time and date are recorded in a memory device of the pump controller 110 (not shown in FIG. 1). In some embodiments, a user may operate the pump controller 110 (either physically or remotely) to turn on the pump 100.

In step 305 a check is made to verify that the electronic sensor 132 indicates that the desired fluid 106 is being pumped. In some embodiments the desired fluid may be oil, water, air, or a mixture thereof. Although this is shown as a single step in the process flow, during operation the electronic sensor 132 (also shown as electronic sensor 232 in FIG. 2), either alone or in combination with sensor controller 130 (also shown as sensor controller 230 in FIG. 2), may continuously monitor if it is submerged in air, oil, water, or a mixture thereof. The determined composition of the fluid 106 in which the electronic sensor 132 is submerged may be provided to pump controller 110. In some embodiments, the determined composition of the fluid 106 is compared to a target fluid that is stored in a memory device of the pump controller 110, to determine if the desired fluid 106 is being pumped.

If the determined composition of the fluid 106 is not the desired fluid, in step 307 the pump controller 100 may control the pump 100 to turn off. For example, if it is determined that the electronic sensor 132 is submerged in water instead of oil, and oil is the desired fluid 106, the pump controller 110 may control the pump 100 to be turned off. This may facilitate the minimization of the amount of water that is pumped when the fluid column in the working tube 102 changes from oil to water and can save significant water disposal costs. If the desired fluid (e.g., oil) is being pumped control is transferred to step 309 and pumping continues.

In step 309 the pumping of the desired fluid 106 continues until the fluid level 126 drops below the electronic sensor 232 (not illustrated in FIG. 1). When the electronic sensor 132 senses the absence of fluid 106, a signal is sent to the sensor controller 130. If the absence of oil is sensed, the microcontroller 252 (shown in FIG. 2) instructs oil sensor relay 260 (shown in FIG. 2) to open, or if the absence of water is sensed, the microcontroller 252 (shown in FIG. 2) instructs the water sensor relay 262 (shown in FIG. 2) to open. In other embodiments, if the absence of oil is sensed, the microcontroller 252 (shown in FIG. 2) instructs oil sensor relay 260 (shown in FIG. 2) to close, or if the absence of water is sensed, the microcontroller 252 (shown in FIG. 2) instructs the water sensor relay 262 (shown in FIG. 2) to close. A signal indicating the opening (or closing) of one or both of the relays, e.g. oil sensor relay 260 or water sensor relay 262, is transmitted to the pump controller 210 (shown in FIG. 2) over conductor 261 (shown in FIG. 2) or conductor 263 (shown in FIG. 2). The pump controller 110 turns the pump off when the fluid level 106 drops below the sensor 132, and records the time and date in a memory device (not shown in FIG. 1 or 2).

In step 311 after a preset time delay, the oil sensor relay 260 (shown in FIG. 2) and/or the water sensor relay 262 (shown in FIG. 2) are reset. For example, if in step 309 the oil sensor relay 260 (shown in FIG. 2) and/or water sensor relay 262 (shown in FIG. 2) was instructed to open, in step 311 the oil sensor relay 260 and/or water sensor relay 262 may be reset to a closed position. If in step 309 the oil sensor relay 260 (shown in FIG. 2) and/or water sensor relay 262 (shown in FIG. 2) was instructed to close, in step 311 the oil sensor relay 260 and/or water sensor relay 262 may be reset to an open position. The reset of the oil sensor relay 260 (shown in FIG. 2) and/or the water sensor relay 262 (shown in FIG. 2) may occur automatically, by an action of the microcontroller 252, or by any suitable method. This change in relay status is transmitted to the pump controller 110, for example by one or more signals sent over conductor 261 (shown in FIG. 2) and/or conductor 263 (shown in FIG. 2), or by wireless transmission using wireless Bluetooth communications module 254. The pump controller 110, upon receiving the change in relay status, turns the pump 100 back on.

In step 313 the pumping time duration is calculated by the pump controller 110. For example, the pump controller 110 may calculate the pumping time duration by comparing the time and date recorded in step 303 to the time and date recorded in step 309. The pump controller may compare the calculated pumping time duration with a target pumping time range. In some embodiments, the target pumping time range is preset and stored in a memory device (not shown in FIG. 1) of the pump controller 110. In some embodiments, the target pumping time range is input by a user before being stored in the memory device.

In step 315 it is determined whether the pumping time duration is within the target pumping time range. If it is determined that the pumping time duration is within the target pumping time range, the process returns to step 303, described above, and pumping continues.

In step 315 if it is determined that the pumping time duration is outside the target pumping range, step 317 is performed.

In step 317 a new time delay value is calculated, and is programmed into one or both of the oil sensor relay 260 and the water sensor relay 262. The process then returns to step to step 303, and pumping continues.

In some applications, such as when a mixture of oil and water is being pumped, the oil sensor relay 260 and the water sensor relay 262 may be wired in parallel (logical OR) so that as long as either oil or water is present, pumping continues.

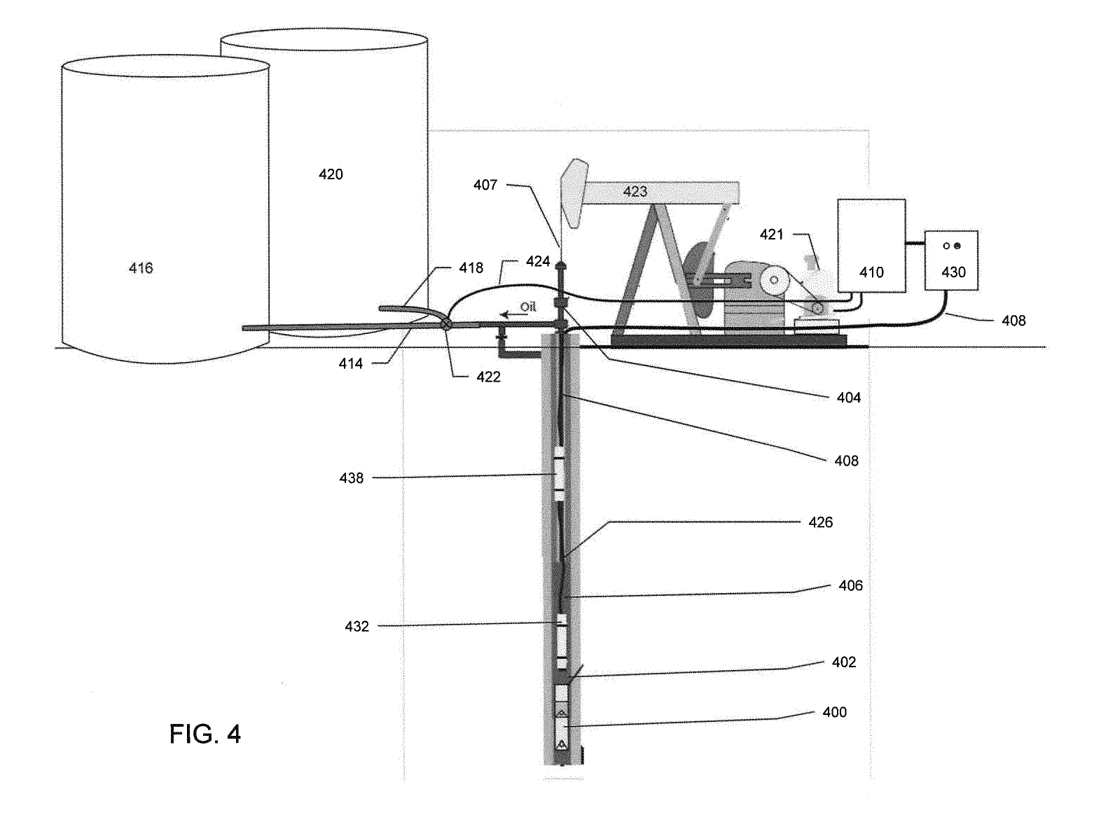

FIGS. 4, 5, and 6 respectively illustrate an AFPS in accordance with some embodiments, a sensor controller which may be used in the embodiment AFPS in accordance with some embodiments, and an embodiment of a method for operating an embodiment AFPS. A pump jack 423 is depicted in FIG. 4 for illustration, but other types of suitable pumps such as an ESP or PCP could also be used.

FIG. 4 shows an oil well and an AFPS in accordance with some embodiments. A first pipe 414 is connected to an oil storage tank 416, which may be used to store oil. A second pipe 418 is connected to a water storage tank 420, which may be used to store water. A valve 422 is operable to direct fluid flow from a wellhead 404 to either oil storage tank 416 or water storage tank 420, depending on the composition of the fluid. In an alternative arrangement an oil/water separator (not shown in FIG. 4), also known as a gun barrel separator, can be positioned between the well head and the oil storage tank 416 and the water storage tank 420. A working tube 402 extends from the wellhead 404 into the oil well. A sucker rod pump 400 is positioned in the well in the working tube 402, and the working tube 402 carries fluid 406 from the sucker rod pump 400 to the well head 404.

A pump controller 410 is disposed at a surface of the well. The pump controller 410 contains electronics to control components of the AFPS depicted in FIG. 4. For example, the pump controller 410 may be configured to receive information from sensors located on equipment of the AFPS, to send power to the pumpjack motor 421, to turn the pumpjack 423 off and on, to control the valve 422, to turn other components on and off, and the like. The pump controller 410 may also contain wireless or Bluetooth communications equipment to enable control from a remote location. Pumpjack rod string 407 is connected between the horsehead of the pumpjack 423 and the sucker rod pump 400, providing the reciprocal up and down motion that works the sucker rod pump 400.

An electrical cable 424 extends between the pump controller 410 and the valve 422. Electrical cable 424 enables the pump controller 410 to send signals to the valve 422 to control the position of the fluid valve 422.

The AFPS includes a sensor system, which includes a lower electronic sensor 432 and an upper electronic sensor 438. The lower electronic sensor 432 is positioned in the well proximate to the pump 400. In some embodiments, the lower electronic sensor 432 is attached to the outside of the working tube 402, about a meter or more above the pump 400. The upper electronic sensor 438 attached to the outside of the working tube 402, a recovery distance above the lower sensor 432. The lower electronic sensor 432 and the upper electronic sensor may be electrically coupled to each other and to sensor controller 430 by cable 408. In other embodiments, lower electronic sensor 432 and the upper electronic sensor may be electrically coupled to each other and/or to sensor controller 430 using multiple cables.

In some embodiments the recovery distance that separates the upper electronic sensor 438 from the lower electronic sensor 432 in the well is determined by the well operator to be the desired height to which the fluid column the well is allowed to recover after the pump 400 is shut off and before the pump 400 is turned back on. For example, when the absence of fluid is detected by the lower electronic sensor 432, a signal may be sent by the lower electronic sensor 432 to the sensor controller 430 using sensor cable 408. The sensor controller 430 receives the signal, and then sends a signal to the pump controller 410, and the pump controller 410 turns the pumpjack motor 421 off, which causes the pump 400 to turn off. When the pump 40o is shut off, the height of the fluid column in the working tube 402 may begin to recover as fluid enters the well through perforations in the well casing. When the upper electronic sensor 438 senses the presence of fluid 406 a signal is sent to the sensor controller 430, which in turn sends a signal to the pump controller 410. Upon receiving the signal from the sensor controller 430, the pump controller 410 turns the pumpjack motor 421 back on, and pumping resumes. The lower sensor 432 protects the pump 400 from damage by indicating whether the pump 400 is pumping fluid. This can help the AFPS to turn the pump 400 off when it is detected that the pump 400 is not pumping fluid, thereby reducing or avoiding pump off.

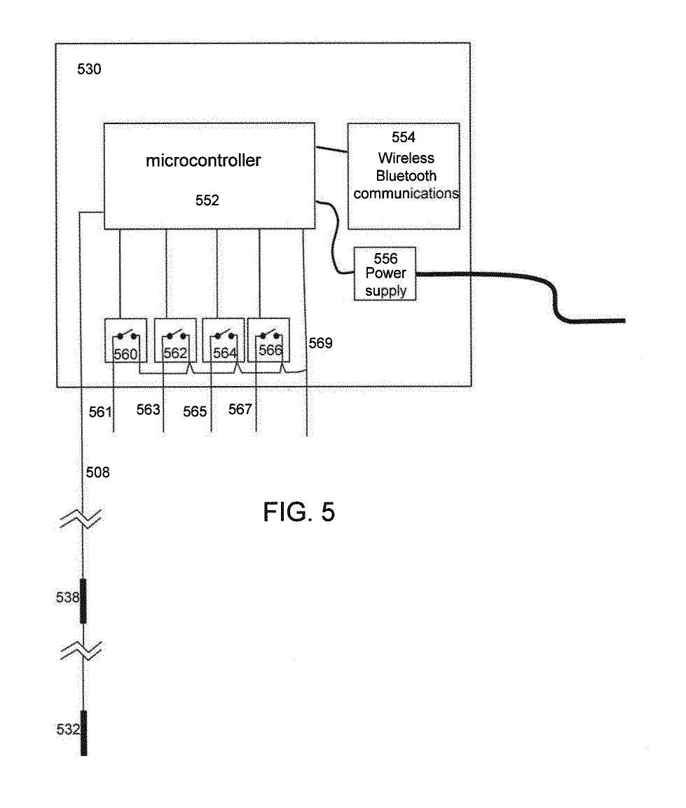

FIG. 5 depicts a sensor controller 530 in accordance with some embodiments. In FIG. 5, elements designated as 5xx correspond to elements designated as 4xx in FIG. 4. For example, sensor controller 530 in FIG. 5 corresponds to the sensor controller 430 in FIG. 4.

As shown in FIG. 5, sensor controller 530 includes microcontroller 552, power supply 556, an optional wireless Bluetooth communications module 554, and relays 560, 562, 564, and 566. Although four relays are shown in FIG. 5, in other embodiments more or less relays may be used. Sensor controller 530 is connected to an upper electronic sensor 538 (depicted as upper electronic sensor 438 in FIG. 4) and lower electronic sensor 532 (depicted as lower electronic sensor 432 in FIG. 4) by cable 508. Power supply 556 provides power to microcontroller 552. In some embodiments, power supply 556 may provide low voltage power to the microcontroller 552. The low voltage power is less than about 30V in some embodiments. The microcontroller 552 receives signals from the lower electronic sensor 532 and the upper electronic sensor 538 through cable 508. The relays 560, 562, 564, and 566 may be designated as oil sensing relays or water sensing relays, and/or may be designated to correspond to one of lower electronic sensor 532 and upper electronic sensor 538 in some embodiments. For example, relay 560 may indicate whether lower electronic sensor 532 detects oil, relay 562 may detect whether upper electronic sensor 538 detects oil, relay 564 may detect whether lower electronic sensor 532 detects water, and relay 566 may detect whether upper electronic sensor 538 detects water. Each of relays 560, 562, 564, and 566 are connected to the microcontroller 552 and also to the pump controller 510. Relay 560 is connected to pump controller 510 by conductor 561, relay 562 is connected to pump controller 510 by conductor 563, relay 564 is connected to pump controller 510 by conductor 565, and relay 566 is connected to pump controller 510 by conductor 567. A conductor 569, for example a ground conductor, may also be used to connect the sensor controller 530 directly to the pump controller 510.

Alternatively, information regarding the status of the relays 560, 562, 564, and 566 can be transmitted wirelessly to the pump controller 510 using the wireless or Bluetooth communications module 554. In which case, conductors 561, 563, 565, 567, and 569 may be unnecessary.

In some embodiments, such as when an oil and water mixture is being pumped, the relays that are designated to indicate oil (for example relay 560 and relay 562) and the relays that are designated to indicate water (for example relay 564 and relay 566) can be connected in parallel so that pumping continues if either water or oil is present.

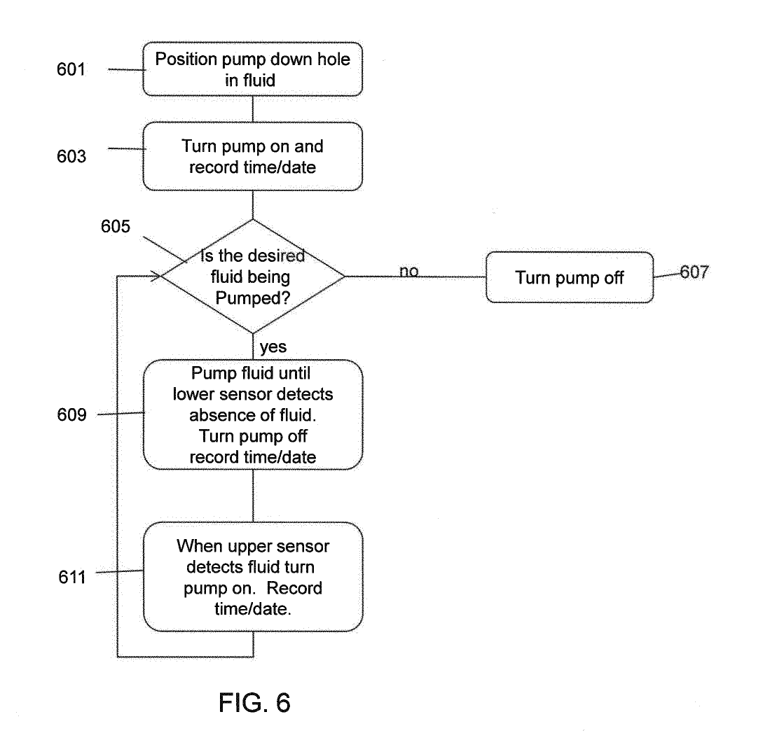

An embodiment of a process of operating of an automated oil recovery system, for example the system shown in FIG. 4, is illustrated in the flow diagram in FIG. 6. In describing the process of FIG. 6, the referenced components are shown in FIG. 4 unless otherwise indicated.

In step 601 the sucker rod pump 400 (or other suitable pump) is submersed in the fluid 406 down hole in the well.

Next, in step 603 the pump is turned on and the time and date are recorded. For example, the pump controller 410 may automatically turn on the pump 400 and the time and date are recorded in a memory device of the pump controller 410 (not shown in FIG. 4). In some embodiments, a user may operate the pump controller 410 (either physically or remotely) to turn on the pump 100.

Next, in step 605 a check is made to see if the desired fluid 406 is being pumped. The desired fluid 406 can be oil, water, or an oil and water mixture. For example, in some embodiments the lower electronic sensor 432 (also shown as lower electronic sensor 532 in FIG. 5), and/or the upper electronic sensor 438 (also shown as upper electronic sensor 538 in FIG. 5), either alone or in combination with sensor controller 430 (also shown as sensor controller 530 in FIG. 5), may continuously monitor whether they are submerged in air, oil, water, or a fluid mixture. The determined composition of the fluid 406 in which the lower electronic sensor 432 and/or upper electronic sensor 438 is submerged may be provided to pump controller 410, which checks to see if the desired fluid 406 is being pumped. In some embodiments, the determined composition of the fluid 406 is compared to a target fluid that is stored in a memory device of the pump controller 410, to determine if the desired fluid 406 is being pumped.

If the result of the determination in step 605 is that the desired fluid 406 is not being pumped, in step 607 the pump is turned off. For example, the pump controller 410 instructs the pumpjack motor 421 to turn off the pump 400.

If the result of the determination in step 605 is that the desired fluid 406 is being pumped, step 609 is performed and pumping continues.

Although the fluid check of step 605 is shown as a single step in FIG. 6, in some embodiments the lower electronic sensor 432 and/or the upper electronic sensor 438 continuously monitor whether they are in air, oil, or water, and immediately send a signal to the sensor controller 430 when any change occurs.

The pumping occurs until the fluid level between the outside of the working tube 402 and the well casing drops. When the fluid level drops below the lower electronic sensor 432, in step 609 the lower electronic sensor 432 senses the absence of fluid 406. Upon detecting the absence of fluid 406, the lower electronic sensor 432 sends a signal to the sensor controller 430. If the detected absence of fluid is a detected absence of oil, microcontroller 552 (shown in FIG. 5) opens relay 560 (shown in FIG. 5). If the detected absence of fluid is a detected absence of water, microcontroller 552 (shown in FIG. 5) opens relay opens relay 564 (shown in FIG. 5). In another embodiment, microcontroller 552 (shown in FIG. 5) may close relay 560 (shown in FIG. 5) or relay 564 (shown in FIG. 5) when an absence of a fluid is detected. The opening (or closing) of one of relay 560 (shown in FIG. 5) or relay 564 (shown in FIG. 5) is transmitted to the pump controller 510 (shown in FIG. 5), for example over conductor 561 (shown in FIG. 5) or conductor 565 (shown in FIG. 5), or wirelessly using wireless Bluetooth communications module 564 (shown in FIG. 5). The pump controller 410 receives the transmitted signal, and then turns the pump 400 off. A time and date of turning the pump off maybe recorded by the pump controller 410 in a memory device of the pump controller (not shown in FIG. 4).

As described above, when pump 400 is turned off a fluid column may begin to recover, for example because fluid enters the well through perforations in the well casing. Eventually, the fluid column will recover to the extent that the upper electronic sensor 438 will become submerged in fluid 406. In step 611, when the fluid column recovers and is detected by the electronic upper sensor 438 (also shown as upper electronic sensor 538 in FIG. 5) the upper electronic sensor 538 sends a signal to microcontroller 552 (shown in FIG. 5). The microcontroller 552 closes (or opens) relay 562 (if the detected fluid is oil) or relay 566 (if the detected fluid is water). The microcontroller 552 also closes (or opens) relay 560 or relay 564, which correspond to the lower electronic sensor 432. This change in status of the relays is transmitted to the pump controller 410 (FIG. 4), for example over conductor 561 (shown in FIG. 5) or conductor 565 (shown in FIG. 5), or wirelessly using wireless Bluetooth communications module 564 (shown in FIG. 5). The pump controller 410 then turns the pump 400 back on.

The process then returns to step 605, and the process continues as described above.

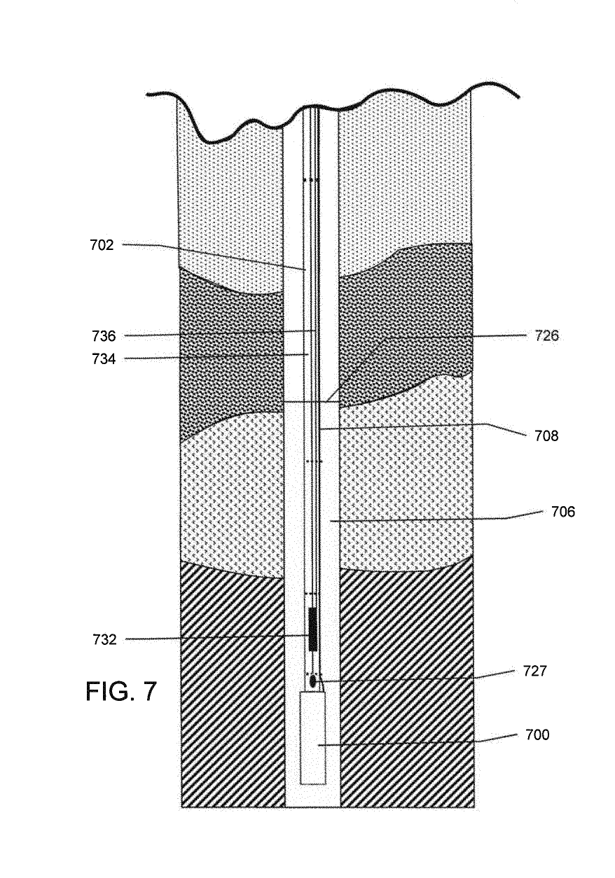

FIG. 7 depicts a down hole portion of another AFPS in accordance with some embodiments. The surface equipment is the same or similar to the surface equipment as described above in connection with FIG. 1 or in connection with FIG. 4 (or a combination thereof) depending upon whether an ESP, PCP, or pumpjack is used. An ESP 700 is used in FIG. 7 for illustration.

The ESP 700 is attached to the lower end of working tube 702. The working tube 702 may be the same as, or similar to, working tube 102 described in connection with FIG. 1 and working tube 402 described in connection with FIG. 4. The ESP 700 pumps fluid through the working tube 702 to the well head (not shown in FIG. 7). A fluid column 706 partially fills the well, and an upper surface of the fluid column 706 defines a fluid/air interface 726. The fluid/air interface 726 is shown at a particular location in FIG. 7, but during operation the fluid/air interface may be lower or higher than illustrated, depending on conditions in the well and operation of the equipment of the AFPS.

An electronic sensor 732 is disposed between the working tube 702 and the well casing, and may be connected to a sensor controller (not shown) by electronic sensor conductor 734. In some embodiments the electronic sensor 732 detects when the fluid/air interface 726 passes the electronic sensor 732 (e.g. from higher than the electronic sensor 732 to lower than the electronic sensor 732, or vice versa). When this occurs, a signal is sent from the electronic sensor 732 to the sensor controller (not shown in FIG. 7) via electronic sensor conductor 734. A pressure sensor 727 may be positioned near the electronic sensor 732 in some embodiments. Pressure sensor 727 may send a signal to the sensor controller (not shown in FIG. 7) using a pressure sensor wire 736. Pressure sensor wire 736 may extend from pressure sensor 727 to the sensor controller (not shown in FIG. 7). Although in FIG. 7 the pressure sensor 727 is shown as a standalone sensor, in some embodiments the pressure sensor 727 can be integrated with the electronic sensor 732 as a fluid/pressure sensor. Additional sensors to detect other parameters such as temperature, conductivity, amperage, can also be integrated with fluid sensor 732.

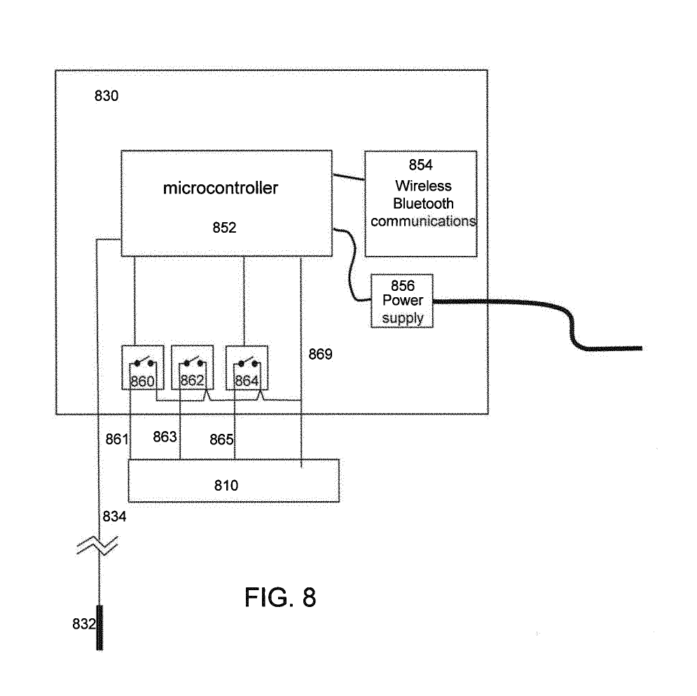

FIG. 8 depicts a sensor controller 830 in accordance with some embodiments. In FIG. 8, elements designated as 8xx correspond to elements designated as 1xx in FIG. 1. For example, sensor controller 830 in FIG. 8 corresponds to the sensor controller 130 in FIG. 1.

As shown in FIG. 8, sensor controller 830 includes microcontroller 852, power supply 856, wireless Bluetooth communications module 854, and relays 860, 862, and 864. Although three embodiments are shown in FIG. 8, in other embodiments more or less relays may be used. Cable 834 connects the sensor controller 830 to one or more of electronic sensors 832 (shown as electronic sensor 732 in FIG. 7) and/or pressure sensor 727 (shown in FIG. 7 only). Although FIG. 7 depicts electronic sensor 732 and pressure sensor 727 being electrically connected to a sensor controller using separate conductors, in some embodiments electronic sensor 732 and pressure sensor 727 maybe connected to sensor controller 830 using a same cable 834. Power supply 856 provides power to microcontroller 852. In some embodiments, power supply 856 may provide low voltage power to the microcontroller 852. The low voltage power may be less than about 30 V in some embodiments.

The microcontroller 852 receives signals from the electronic sensor 732 and the pressure sensor 727, for example through cable 834. Relays 860, 862, and 864 are each electrically connected to (and controlled by) microcontroller 852. Relay 860 is connected to pump controller 810 by conductor 861. Relay 862 is connected to pump controller 810 by conductor 863. Relay 864 may be connected to pump controller 810 by conductor 865. A conductor 869 may also be used to directly connect the sensor controller 830 to the pump controller 810. In some embodiment, relay 860 may be used to indicate the detection of oil by electronic sensor 832, relay 862 may be used to indicate the detection of water by electronic sensor 832, and relay 864 may be used to indicate a detection of the pressure sensor 272 (shown in FIG. 7).

In some embodiments, information regarding the status of the oil, water, and pressure can be transmitted wirelessly to the pump controller 810 using a wireless or Bluetooth communications module 854. In this case, conductor 861, conductor 863, 865, and/or conductor 869 may not be necessary.

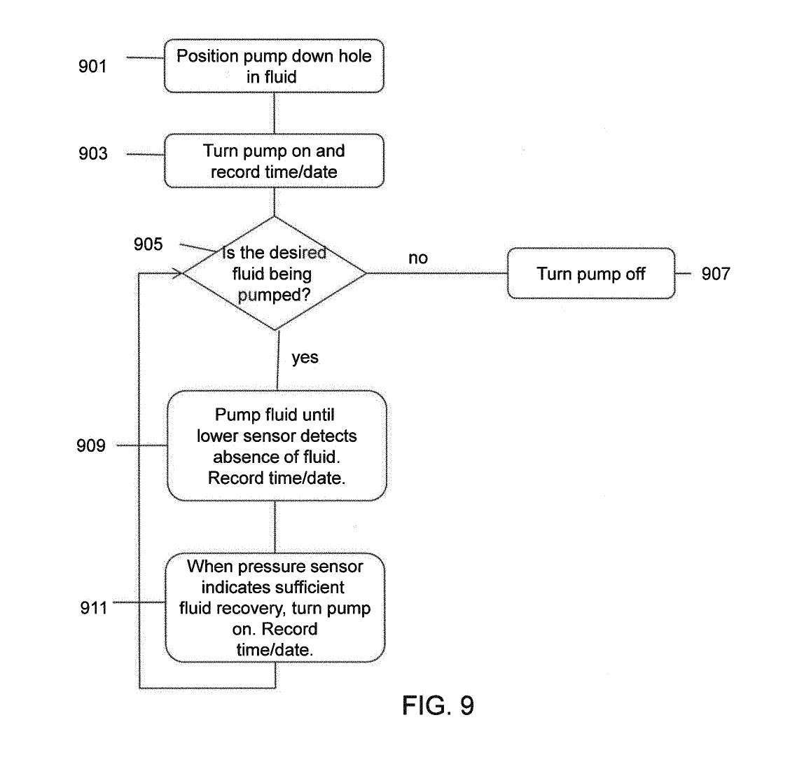

The operation of an automated oil recovery system, for example as shown in FIG. 7, is illustrated in the flow diagram in FIG. 9 in accordance with an embodiment. In describing the process of FIG. 9, the referenced components are shown in FIG. 7 unless otherwise indicated.

In step 901 the pump 700 is submersed in the fluid 706 down hole in the well.

Next, in step 903 the pump 700 is turned on and the time and date are recorded. For example, the pump controller 110 (shown in FIG. 1) may automatically turn on the pump 700 and the time and date are recorded in a memory device of the pump controller 110 (not shown in FIG. 7 or 1). In some embodiments, a user may operate the pump controller 110 (shown in FIG. 1), either physically or remotely, to turn on the pump 700.

Next, in step 905 a check is made to see if the desired fluid 706 is being pumped. The desired fluid 706 can be oil, water, or an oil and water mixture. For example, in some embodiments the electronic sensor 732 (also shown as electronic sensor 832 in FIG. 8), either alone or in combination with sensor controller 830 (shown in FIG. 8), may continuously monitor whether it is submerged in air, oil, water, or a fluid mixture. The determined composition of the fluid 706 in which the electronic sensor 732 is submerged may be provided to pump controller 110 (shown in FIG. 1), which checks to see if the desired fluid 706 is being pumped. In some embodiments, the determined composition of the fluid 706 is compared to a target fluid that is stored in a memory device of the pump controller 110, to determine if the desired fluid 706 is being pumped.

If the result of the determination in step 905 is that the desired fluid is not being pumped, step 907 is performed and the pump 700 is turned off. For example, the oil pump controller 110 (shown in FIG. 1) may control pump 700 to turn off.

If the result of the determination in step 905 is that the desired fluid 706 is being pumped, pumping continues. As the pumping continues, the fluid level in the well may begin to drop. In step 909, it is detected that the fluid level in the well has dropped to an extent that the electronic sensor 732 is not submerged in the fluid 706.

Although the checking step of step 909 is shown as a single step in this flow diagram, in some embodiments the electronic sensor 732 continuously monitors whether the electronic sensor 732 is in air, oil, or water, or a mixture therof, and sends a signal to the sensor controller 830 (shown in FIG. 8) when any detected change occurs.

In step 909 when it is detected that the fluid level has dropped below the electronic sensor 732, the electronic sensor 732 sends a signal to the sensor controller 830. If the detected absence of fluid is a detected absence of oil, the microcontroller 852 (shown in FIG. 8) may cause relay 860 (shown in FIG. 8) to open. If the detected absence of fluid is a detected absence of water, microcontroller 852 (shown in FIG. 8) may cause relay 862 (shown in FIG. 8) to open. A signal indicating the opening of one of relay 860 (shown in FIG. 8) or relay 862 (shown in FIG. 8) is transmitted to the pump controller 810 (shown in FIG. 8). Upon receiving the signal, pump controller (shown in FIG. 8) may turn the pump 700 off.

Upon turning off the pump 700, the fluid 706 in the well may begin to rise as discussed above. A level of the fluid recovery may be monitored by pressure sensor 727. In step 911 when the fluid 706 recovers to an extent that the pressure sensor 727 senses a target pressure value, the pressure sensor 727 sends a signal to the sensor controller 830 (shown in FIG. 8). For example, the pressure sensor 727 sends a signal to the microcontroller 852 (shown in FIG. 8) and the microcontroller 852 (shown in FIG. 8) closes relay 864 (shown in

FIG. 8),and closes relay 860 (shown in FIG. 8) if oil is present or closes relay 862 (shown in FIG. 8) if water is present. This change in status of the relays is transmitted to the pump controller 810 (shown in FIG. 8). The pump controller 810 (shown in FIG. 8) then turns the pump 700 back on. A time and a date of turning the pump back on may be recorded, for example in a memory device of the pump controller 810.

Next, step 905 is performed, and the process continues as described above.

As described herein, an automated fluid pumping system (AFPS) integrates an electronic fluid sensor and sensor controller with a pump controller. In some embodiments, the electronic fluid sensor is configured to detect the presence or absence of fluid in a well, and/or may be configured to determine the composition of a detected fluid in the well. The electronic fluid sensor may detect the presence or absence of fluid, or the composition of a detected fluid, electronically with no moving parts. In some embodiments, the electronic fluid sensor can detect oil or can detect water, and can determine if the fluid sensor is in water, in oil, in air, and/or a mixture thereof. Additional sensors that provide additional capability such as temperature, pressure, amperage, and conductivity can be integrated with the electronic fluid sensor in some embodiments. The electronic fluid sensor may interact with the pump controller in the AFPS to control the AFPS equipment. In some embodiments, the AFPS may reduce the time a pumper needs to spend at the well, and also protect the pump from damage due to pump off.

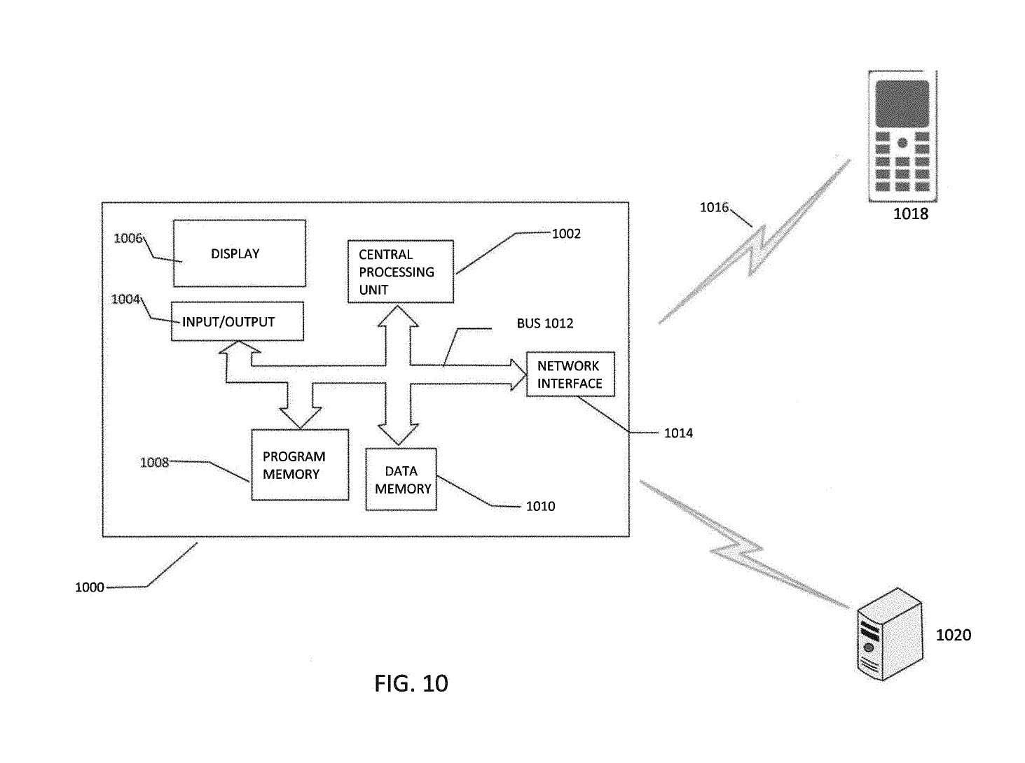

FIG. 10 is a block diagram of elements of a processing system 1000 that may be used to implement a controller or a microcontroller, for example sensor 130 and/or pump controller 110 as described in connection with FIG. 1, microcontroller 252 as described in connection with FIG. 2, sensor controller 430 and pump controller 410 as described in connection with FIG. 4, microcontroller 552 as described in connection with FIG. 5, and/or microcontroller 852 as described in connection with FIG. 8. The processing system 1000 may be equipped with one or more input/output devices 1004, such as a video adapter/graphics processing unit (“GPU”). The processing system 1000 may include a central processing unit (“CPU”) 1002, program memory 1008, data memory 1010, and a hardware accelerator connected to a bus 1012.

The bus 1012 may be one or more of any type of several bus architectures including a memory bus or memory controller, a peripheral bus, video bus, or the like. The CPU 1002 may be formed with any type of electronic data processor. The memory, 1008 and 1010, may be formed with any type of system memory such as static random access memory (SRAM), dynamic random access memory (DRAM) such as a synchronous DRAM (SDRAM), read-only memory (ROM), nonvolatile random access memory (“NVRAM”), a combination thereof, or the like. In an embodiment, the memory may include ROM for use at boot-up, and DRAM for data storage for use while executing programs. The program memory 1008 may store programs, for example programs enabling the processes as described in connection with FIGS. 1-9, including but not limited to the processed depicted in FIGS. 3, 6, and 9.

A video adapter may provide an interface to couple an external input and output from a display 1006 to the processor 1002. Other devices may be coupled to the processing system 1000, and additional or fewer interface cards may be utilized. For example, a serial interface card (not shown) may be used to provide a serial interface for a printer.

The processing system 1000 may also include a network interface 1014, which can be a wired link, such as an Ethernet cable or the like, and/or a wireless link 1016 to enable communication with a network such as a cellular communication network or a Bluetooth communication network. The network interface allows the processor to communicate with remote units via the network. In an embodiment, the processing system 1000 is coupled to a local-area network or a wide-area network to provide communications to remote devices, such as other processors 1020, cell phones 1018, the Internet, remote storage facilities, or the like.

It should be noted that processing system 1000 may include other components. For example, processing system 1000 may include power supplies, cables, a motherboard, removable storage media, cases, and the like. These other components, although not shown, are considered part of processing system 1000.

Modifications are possible in the described embodiments, and other alternative embodiments are possible within the scope of the claims.

What is claimed is:

1. An automated fluid pumping system (AFPS), comprising:
a well head disposed at a surface of a well;
a fluid pump disposed in the well;
working tubing coupled between the fluid pump and the well head;
a first electronic fluid sensor disposed in the well, the first electronic fluid sensor being physically coupled to an outer surface of the working tubing above the fluid pump;
a sensor controller electrically coupled to the first electronic fluid sensor, the sensor controller comprising a first relay, wherein the sensor controller is configured to receive an electronic signal from the first electronic fluid sensor, and to change a state of a first relay from a first state to a second state; and
a pump controller electrically coupled to the sensor controller, wherein the pump controller is configured to control the fluid pump in response to a signal from the sensor controller indicating the change in the state of the first relay.

2. The AFPS of claim 1, wherein the first electronic fluid sensor is configured to be submerged in a fluid, and to determine whether the fluid comprises air, oil, or water.

3. The AFPS of claim 1, further comprising:
a second relay, wherein the sensor controller is configured to change the state of the first relay when the first electronic fluid sensor detects a first fluid, and wherein the sensor controller is configured to change a state of the second relay when the first electronic fluid sensor detects a second fluid.

4. The AFPS of claim 1, further comprising:
a second electronic fluid sensor physically coupled to an outer surface of the working tubing above the first electronic fluid sensor, wherein the second electronic fluid sensor is electrically coupled to the sensor controller and electrically coupled to a second relay.

5. The AFPS of claim 1, further comprising:
a pressure sensor physically coupled to the working tubing proximate to the first electronic fluid sensor, wherein the pressure sensor is electrically coupled to the sensor controller and electrically coupled to a third relay.

6. The AFPS of claim 1, wherein the first electronic fluid sensor comprises an integrated pressure sensor, and the integrated pressure sensor is electrically coupled to a third relay.

7. The AFPS of claim 1, wherein the first electronic fluid sensor comprises an integrated pressure sensor and an integrated temperature sensor.

8. The AFPS of claim 1, wherein the first relay is a time delay relay, and is configured to reset from the second state to the first state after a predetermined time delay.

9. The AFPS of claim 1, wherein the first relay is a reprogrammable time delay relay.

10. A method for operating an automated fluid pumping system (AFPS), comprising:
receiving, by a sensor controller, a first signal from a first electronic fluid sensor, wherein the first signal indicates that the first electronic fluid sensor is submerged in a desired fluid;
in response to the first signal indicating that the first electronic fluid sensor is submerged in the desired fluid, changing, by the sensor controller, a state of a first relay from a first state to a second state;
determining, by a pump controller, that the state of the first relay has changed from the first state to the second state;
in response to determining that that the state of the first relay has changed from the first state to the second state, controlling, by the pump controller, a fluid pump to begin pumping;
receiving, by the sensor controller, a second signal from the first electronic fluid sensor, wherein the second signal indicates that the first electronic fluid sensor is no longer submerged in the desired fluid;
in response to the second signal indicating that the first electronic fluid sensor is no longer submerged in the desired fluid, changing, by the sensor controller, the state of a first relay from the second state to a the first state;
in response to determining that that the state of the first relay has changed from the second state to the first state, controlling, by the pump controller, the fluid pump to stop pumping;
after a time delay, changing, by the sensor controller, the state of the first relay from the first state to the second state, and controlling, by the pump controller, the fluid pump to being pumping.

11. The method of claim 10, wherein the first electronic fluid sensor is disposed in a well, and the desired fluid is oil, water, or an oil and water mixture.

12. The method of claim 10, wherein the first electronic fluid sensor is disposed in a well, and wherein the desired fluid is oil.

13. The method of claim 10, wherein the first electronic fluid sensor is disposed in a well, and wherein the desired fluid is an oil and water mixture.

14. A method for operating an automated fluid pumping system (AFPS), comprising:
receiving, by a sensor controller, a first signal from a first electronic fluid sensor, wherein the first signal indicates the first electronic fluid sensor is submerged in a fluid in a well;
changing, by the sensor controller, a state of a first relay of the sensor controller in response to receiving the first signal;
in response to a pump controller determining the state of the first relay has changed, controlling, by the pump controller, a fluid pump in the well to being pumping;
receiving by the sensor controller, a second signal from the first electronic fluid sensor, wherein the second signal indicates that the first electronic fluid sensor is not submerged in the fluid, and changing the state of the first relay in response to receiving the second signal;
in response to the pump controller determining the state of the first relay has changed, controlling, by the pump controller, the fluid pump to stop pumping, wherein when the fluid pump stops pumping a fluid level in the well begins to recover;
receiving, by the sensor controller, a third signal from a second sensor, and changing a state of a second relay of the sensor controller in response to receiving the third signal from the second sensor; and
determining, by the pump controller, that the state of the second relay has changed, and controlling, by the pump controller, the fluid pump to begin pumping.

15. The method of claim 14, wherein the second sensor is a second electronic fluid sensor, wherein the second electronic fluid sensor is disposed in the well above the first electronic fluid sensor and the third signal is sent when the fluid level in the well rises to an extent that the second electronic fluid sensor is submerged in fluid.

16. The method of claim 14, wherein the second sensor is a pressure sensor proximate to the first electronic fluid sensor, and wherein the third signal is sent by the pressure sensor when a fluid level in the well exerts a preset pressure on the pressure sensor.

17. The method of claim 16, wherein the pressure sensor and the first electronic fluid sensor are integrated together.

18. The method of claim 17, wherein the fluid is oil or water.

19. The method of claim 17, wherein the fluid is oil.

20. The method of claim 17, wherein the fluid is an oil and water mixture.
